(19) United States
(12) Patent Application Publication
(10) Pub. No.: US 2021/0222144 A1
(43) Pub. Date: Jul. 22, 2021

(54) SELECTIVE HYDRATION OF OLEIC ACID DERIVATIVES

(71) Applicant: DSM IP Assets B.V., Heerlen (NL)

(72) Inventors: Harald PICHLER, Graz (AT); Martin SCHÜRMANN, Geleen (NL); Rolf SCHUETZ, Kaiseraugst (NL); Matthias ENGLEDER, Graz (AT); Gernot STROHMEIER, Graz (AT)

(21) Appl. No.: 15/734,370

(22) PCT Filed: Jun. 4, 2019
(86) PCT No.: PCT/EP2019/064525

§ 371 Date: Dec. 2, 2020

(30) Foreign Application Priority Data
Jun. 4, 2018 (CH) 00706/18

(51) Int. Cl. C12N 9/88; C11C 3/00

(52) U.S. Cl. CPC C12N 9/88; C12Y 402/01053; C11C 3/00

(57) ABSTRACT

The present invention relates to regio- and stereoselective hydration of oleic acid derivatives, including esters of oleic acid, by action of modified oleate hydratase.

The present invention relates to regio- and stereoselective hydration of oleic acid derivatives, including esters of oleic acid, by action of modified oleate hydratase.

Fatty acid hydratases (FAHYs; EC 4.2.1.-) catalyze the formation of medium- and long-chain hydroxy fatty acids by addition of water to isolated carbon-carbon double bonds of free mono- or polyunsaturated fatty acids. As such, they provide access to secondary and tertiary alcohols, which makes them valuable tools for the production of a variety of chemicals, including flavor additives, cosmetics, surfactants, lubricants and precursors in polymer chemistry. The use of FAHYs in synthesis promises obvious advantages relating to their exquisite regio- and stereoselectivity, which permits reactions that are not possible with the unselective acid-catalyzed chemical hydration. Most known FAHYs are highly regioselective in hydrating either the cis-9 or cis 12 double bond(s) of unsaturated fatty acids. There is nothing known on the hydration of fatty acid esters by any of the known FAHYs.

The most thoroughly characterized FAHY to date is the oleate hydratase from Elizabethkingia meningoseptica (OhyA, EC 4.2.1.53). It catalyzes the regio- and stereoselective hydration of oleic acid (OA), yielding (R)-10-hydroxy stearic acid with an excellent enantiomeric excess (ee) of at least 98% and without the need for co-factor recycling.

Until now, the applicability of FAHYs in an industrial setting is still limited, which can be mostly attributed to their narrow substrate scope: all work demonstrated that the carboxylic group, a double bond in cis-conformation, a minimum distance of 7 carbons between the carboxylate and the cis-double bond and a minimum fatty acid chain length of 11 carbons are mandatory for conversion. Hydration at a terminal carbon has also not been described so far, as the formation of a partial positive charge at the primary carbon would be in conflict with the proposed reaction mechanism.

Thus, there is a need to broaden the substrate spectrum of FAHYs, in particular OhyA, as the best enzyme characterized so far, in order to use it as a tool in industrial processes. The excellent regio- and stereoselectivity should however not be affected.

Surprisingly, we now found that the substrate tolerance of OhyA towards oleic acid derivatives is triggered by the modification of certain amino acids located in the active side cavity (substrate binding region), i.e. highly conserved amino acid residues (according to sequence alignment in the Hydratase Engineering Database HyED; see Table 1).

Particularly, the present invention is directed to modified enzymes having the activity of FAHYs (EC 4.2.1.-), such as e.g. the activity of OhyA (EC 4.2.1.53), said enzyme catalyzing hydration reactions with an ee of at least 98%, wherein the modified enzyme comprises one or more amino acid substitution(s) at a position corresponding to residues selected from the group consisting of position 265, 436, 438, 442, and combinations thereof in the polypeptide according to SEQ ID NO:1.

The polypeptide according to SEQ ID NO:1, showing OhyA activity, including a polypeptide encoded by a polynucleotide according to SEQ ID NO:2, has been isolated from Elizabethkingia meningoseptica (sequence derived from GenBank accession ACT54545.1).

FAHYs have been isolated from different origins, including mammals, yeast or plants, or bacteria. As used herein, a “modified” enzyme, i.e. modified FAHY, particularly modified OhyA, has a preferred activity and/or specificity towards regio- and stereoselective hydration of oleic acid derivatives compared to a non-modified enzyme. A “non-modified” FAHY, particularly non-modified OhyA, as used herein refers to the respective enzymes not carrying one or more amino acid substitution(s) as defined herein, also referred to herein as wild-type enzymes.

As used herein, a host cell carrying a modified FAHY activity as defined herein, particularly OhyA comprising one or more amino acid substitution(s) as defined herein, is referred to as “modified” host cell. The respective host cell carrying a non-modified enzyme activity, i.e. encoding the wild-type OhyA gene, is referred to as “non-modified” host cell.

In one embodiment, the modified enzyme as defined herein, in particular modified OhyA activity, comprises an amino acid substitution at a position corresponding to residue 265 in the polypeptide according to SEQ ID NO:1, preferably substitution of glutamine by alanine (Q265A). Preferably, the enzyme having modified OhyA activity is originated from E. meningoseptica. The mutation might be combined with 1, 2, 3 or more mutations as defined herein.

In one embodiment, the modified enzyme as defined herein, in particular modified OhyA activity, comprises an amino acid substitution at a position corresponding to residue 436 in the polypeptide according to SEQ ID NO:1, preferably substitution of asparagine by alanine (T436A). Preferably, the enzyme having modified OhyA activity is originated from E. meningoseptica. The mutation might be combined with 1, 2, 3 or more mutations as defined herein.

In a further embodiment, the modified enzyme as defined herein, in particular modified OhyA activity, comprises an amino acid substitution at a position corresponding to residue 438 in the polypeptide according to SEQ ID NO:1, preferably substitution of asparagine by alanine (N438A). Preferably, the enzyme having modified OhyA activity is originated from E. meningoseptica. The mutation might be combined with 1, 2, 3 or more mutations as defined herein.

In a further embodiment, the modified enzyme as defined herein, in particular modified OhyA activity, comprises an amino acid substitution at a position corresponding to residue 442 in the polypeptide according to SEQ ID NO:1, preferably substitution of histidine by alanine (H442A). Preferably, the enzyme having modified OhyA activity is originated from E. meningoseptica. The mutation might be combined with 1, 2, 3 or more mutations as defined herein.

Preferably, the amino acid substitution at a position corresponding to residue Q265A in SEQ ID NO:1 might be combined with further substitutions, such as amino acid substitutions at position(s) corresponding to T436A in SEQ ID NO:1 and/or N438A in SEQ ID NO:1 and/or H442A in SEQ ID NO:1. A preferred modified enzyme is an enzyme having OhyA activity and comprises at least an amino acid substitution at a position corresponding to Q265A, T436A, N438A in SEQ ID NO:1, showing at least 2-fold increase in the conversion of the respective oleate derivative (see FIGS. 3 and 4).

When using hydroxamic acid as substrate, at least 5-fold enhanced relative hydration activity by applying modified OhyA variants Q265A/N438A and OhyA Q265A/T436A/N438A in bioconversions could be achieved. Using oleyl alcohol, i.e. substrate (5), hydration with OhyA Q265A/T436A/N438A was at least 2-fold higher compared to using the wt-OhyA. The relative activities for methyl oleate, substrate (6), and ethyl oleate, substrate (7), were 6-fold higher, and for the n-propyl oleate, substrate (9), even 20-fold higher when using the triple mutation.

As used herein, the activity of OhyA is modified. This might be achieved by, e.g. introducing (a) mutation(s) into the gene coding for OhyA, i.e. amino acid substitution(s) on one or more positions as described herein. The skilled person knows how to genetically manipulate a cell resulting in modification OhyA activity. These genetic manipulations include, but are not limited to, e.g. gene replacement, gene amplification, gene disruption, transfection, transformation using plasmids, viruses, or other vectors.

The generation of a mutation into nucleic acids or amino acids, i.e. mutagenesis, may be performed in different ways, such as for instance by random or side-directed mutagenesis, physical damage caused by agents such as for instance radiation, chemical treatment, or insertion of a genetic element. The skilled person knows how to introduce mutations.

The present invention is particularly directed to the use of such modified OhyA enzymes as defined herein in a process for conversion of non-natural oleic acid derivatives. Preferably, the modified enzymes of the present invention are introduced and/or expressed in a suitable host cell, such as E. coli, i.e. expressed as recombinant or heterologous enzymes.

The conversion reactions might be used with cell-free extracts (CFEs) from E. coli containing recombinantly expressed modified OhyA as defined herein or with E. coli whole cells in a biotransformation reaction. Preferably, the conversion of the substrates as defined herein are performed in biotransformation reactions, such as e.g. using E. coli cells harboring recombinant OhyA upon a 96-h biotransformation. Products from biotransformation might be purified and verified by NMR analysis.

With the modified enzymes as described herein, various non-natural oleic acid derivatives could be hydrated, leading to an ee of at least 98, such as 99 or even 100%.

In one aspect, the present invention is directed to a process for conversion of a non-natural fatty acid derivative, i.e. hydration reaction of fatty acids lacking a free carboxylate head group, using a modified enzyme as defined herein, wherein an ee or at least 98% is achieved. Preferably, the process is independent of any known co-substrate or co-factors such as oxidized or reduced FAD or dithiothreitol (DTT). Preferably, the process is conducted in whole cell biotransformation for at least 22 h.

As used herein, the term “oleic acid derivative” refers to non-natural fatty acid derivatives lacking a free carboxylate head group used as substrate for the OhyA-enzymes described herein. It includes but is not limited to short-chain oleate esters, such as e.g. methyl oleate, ethyl oleate, i-propyl oleate, n-propyl oleate, n-butyl oleate or amides, such as e.g. oleamide or N—OH oleamide, hydroxamic acid, or alcohols, such as e.g. oleyl alcohol. Preferably, it includes substrates (1) to (10) as defined herein, such as oleic acid (1), oleyl amine (2), oleamide (3), N—OH oleamide (4), oleyl alcohol (5), OA methyl ester (6), OA ethyl ester (7), OA i-propyl ester (8), OA n-propyl ester (9), or OA n-butyl ester.

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a suitable (cell-free) system in the presence of a suitable substrate. The skilled person knows how to measure enzyme activity, such as e.g. by HPLC.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code).

### FIGURES

FIG. 1. UV-Vis absorption spectra of purified OhyA before (black curve) and after (orange curve) reconstitution of the flavoprotein, as well as after reduction of the FAD cofactor with DTT (blue curves). UV-Vis absorption spectra of wt-OhyA (FIG. 1A). UV-Vis absorption spectra of mutant OhyA Q265A/T436A/N438A (FIG. 1B). For more explanation see text.

FIG. 2. Conversion of oleamide (FIG. 2A), N-hydroxy oleamide (FIG. 2B), oleyl alcohol (FIG. 2C), OA methyl ester (FIG. 2D), OA ethyl ester (FIG. 2E), OA isopropyl ester (FIG. 2F), OA n-propyl ester (FIG. 2G), OA n-butyl ester (FIG. 2H) by OhyA wild type (WT) and the amino acid exchange variants as whole cell E. coli biocatalysts after over-expression of the enzymes. Control reactions contained either the substrate added to the reaction buffer without cells or the substrate added to an E. coli empty vector control (EVC).

FIG. 3. Regio- and stereoselective hydration of oleic acid (OA), i.e. substrate (1), and OA derivatives, i.e. substrates (2) to (10), by E. meningoseptica mutant oleate hydratase (OhyA). A whole cell E. coli biocatalyst harboring the over-expressed hydratase was used in the biotransformation assays. For more explanation see text.

FIG. 4. Conversion of oleic acid, substrate (1), and non-natural oleic acid derived substrates (2) to (10) by OhyA wild type (WT) and OhyA Q265A/T436A/N438A. The reactions were performed for 22 h using a whole cell E. coli biocatalyst upon expressing of the enzymes.

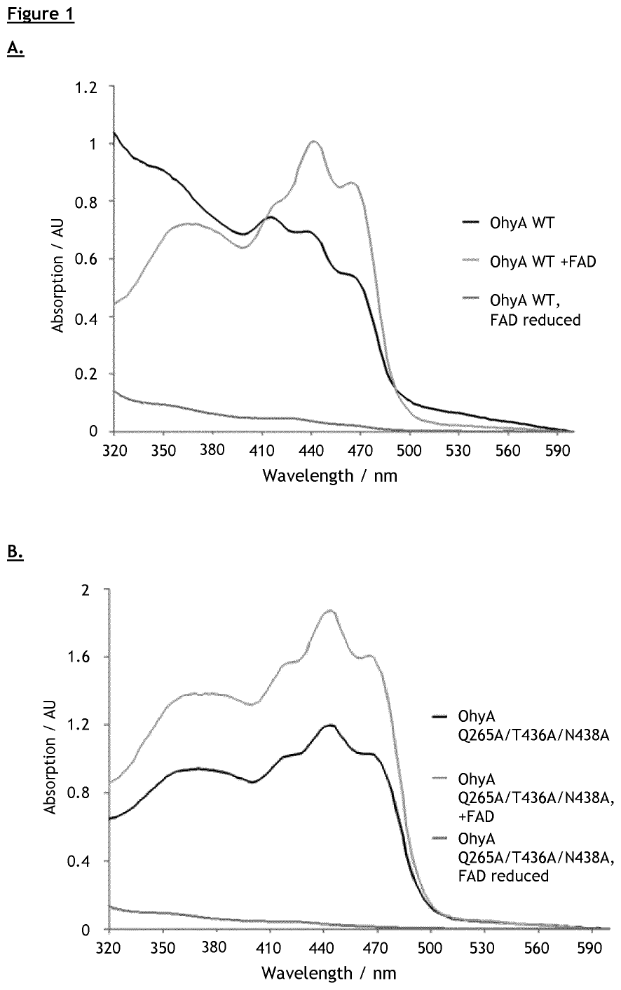

The following examples are illustrative only and are not intended to limit the scope of the invention in any way.

### EXAMPLES

### Example 1: General Methods, Strains and Plasmids

Unless stated otherwise, standard laboratory reagents were obtained from Sigma-Aldrich® (Steinheim, Germany) or Carl Roth GmbH & Co. KG (Karlsruhe, Germany) with the highest purity available. Oleic acid (OA) and esters thereof (methyl, ethyl, i-propyl ester), oleyl alcohol and oleyl amine were purchased from Sigma-Aldrich® (Steinheim, Germany). The OA methyl and ethyl ester and oleyl alcohol were distilled prior to use to a purity >90% according to GC-FID analysis.

Molecular cloning of the expression vector was performed according to standard procedures (Ausubel et al., Current Protocols in Molecular Biology, 2003). and correct integration of the insert was confirmed by sequencing (LGC Genomics, Berlin, Germany). For gene amplification, Phusion® High Fidelity DNA polymerase (Thermo Fisher Scientific Inc., St. Leon-Rot, Germany) was utilized in accordance with the recommended PCR protocol. A codon-optimized gene variant of OhyA (Elizabethkingia meningoseptica XP_001209325 oleate hydratase) was purchased from DNA2.0 (Menlo Park, Calif.). For expression of recombinant OhyA, a modified pMS470 expression vector, pMS470-HISTEV-OhyA was constructed. For all cloning steps and plasmid replication, E. coli Top10 F′ (F[lacI^{q} Tn10(tetR)] mcrA Δ(mrr-hsdRMS-mcrBC) φ80lacZΔM15 ΔlacX74 deoR nupG recA1 araD139 Δ(ara-leu)7697 galU galK rpsL(Str^{R}) endA1λ⁻) from Life technologies (Vienna, Austria) was used. Recombinant OhyA was expressed in E. coli BL21Star™ (DE3) (F- ompT hsdS_{B} (r_{B}⁻m_{B}⁻) gal dcm rne131 (DE3)) (Life technologies, Vienna, Austria).

The protein sequence of OhyA was compared to the amino acid sequences in the Hydratase Engineering Database (HyED), in which a total of 2046 sequences are collected. Since OhyA is categorized in homologous family 11 (HFam11) of the HyED, all amino acid sequences from HFam11 were selected for the multiple sequence alignment. Sequences were extracted from the database for a multiple sequence alignment with the Clustal Omega sequence alignment tool using default settings as described in Sievers et al. (Mol. Syst. Biol. 7, 539, 2011), and were visualized with the UGene software. The positions of highly conserved residues which were used for construction of the OhyA mutants are listed in Table 1.

TABLE 1
multiple sequence alignment of amino acid sequences from HFam11
collected in the hydratase engineering database (HyED), highlighting
the conserved residues involved in binding of a carboxylate.
Note that H442 is conserved among all but one member, where it
is substituted with a Q. For more explanation see text.

| | | | | |
|---|---|---|---|---|
| Elizabethkingia meningoseptica | Q265 | T436 | N438 | H442 |
| Methylobacterium extorquens | Q257 | T428 | N431 | H435 |
| Methylobacterium sp. MB200 | Q257 | T428 | N431 | H435 |
| Bradyrhizobium elkanii | Q256 | T427 | N429 | H433 |
| Bradyrhizobium sp DFCI-1 | Q256 | T427 | N429 | H433 |
| Stenotrophomonas maltophilia | Q261 | T432 | N434 | H438 |
| Stenotrophomonas rhizophila | Q261 | T432 | N434 | H438 |
| Sphingobium yanoikuyae | Q263 | T434 | N436 | H440 |
| Acinetobacter ursingii | Q265 | T436 | N438 | H442 |
| Idiomarina loihiensis | Q263 | T434 | N436 | H440 |
| Pseudomonas pelagia | Q263 | T434 | N436 | H440 |
| Sphingomonas ssp. NM-1 | Q255 | T426 | N428 | H432 |
| Haematobacter missouriensis | Q259 | T430 | N432 | H436 |
| Rhodopseudomonas palustris | Q260 | T431 | N433 | H437 |
| Aurelmonas altamirensis | Q256 | T427 | N429 | H433 |
| Pseudoalteromonas haloplanktis | Q255 | T426 | N428 | H432 |
| Paracoccus ssp. 5503 | Q257 | T428 | N430 | H434 |
| Paracoccus ssp. 10990 | Q257 | T428 | N430 | H434 |
| Paracoccus aminophilus | Q253 | T424 | N426 | H430 |
| Marinomonas prodfundimaris | Q263 | T434 | N436 | H440 |
| Marinomonas posidonia | Q263 | T434 | N436 | H440 |
| Bermanella marisrubri | Q258 | T429 | N431 | H435 |
| Pleomorphomonas koreensis | Q256 | T427 | N429 | H433 |
| Comamonas testosteroni | Q262 | T433 | N435 | H439 |
| Enhydrobacter aerosaccus | Q263 | T434 | N436 | Q440 |
| Hyphomonas beringensis | Q263 | T434 | N436 | H440 |
| Hyphomonas adhaerens | Q263 | T434 | N436 | H440 |
| Gluconobacter oxydans | Q260 | T431 | N433 | H437 |
| Mesonia mobilis | Q265 | T436 | N438 | H442 |
| Sphingobacterium sp. Ag1 | Q265 | T436 | N438 | H442 |
| Flavobacterium psychrophilum | Q265 | T436 | N438 | H442 |
| Flavobacterium hibernum | Q265 | T436 | N438 | H442 |
| Flavobacterium hydatis | Q265 | T436 | N403 | H407 |
| Sulfurospirillum multivorans | Q268 | T439 | N441 | H445 |
| Chryseobacterium sp. JM1 | Q265 | T436 | N438 | H442 |
| Chryseobacterium taiwanense | Q265 | T436 | N438 | H442 |
| Chryseobacterium soli | Q265 | T436 | N438 | H442 |
| Chryseobacterium vrystaatense | Q265 | T436 | N438 | H442 |
| Halomonas ssp. TD01 | Q270 | T441 | N443 | H447 |
| Oleispira antarctica | Q245 | T416 | N418 | H422 |
| Marinobacter sp. HL-58 | Q270 | T441 | N443 | H447 |
| Marinobacter santoriniensis | Q270 | T441 | N443 | H447 |
| Marinobacter excellens | Q270 | T441 | N443 | H447 |
| Psychroflexus torquis | Q265 | T436 | N438 | H442 |
| Olivibacter sitiensis | Q265 | T436 | N438 | H442 |
| Cellulophaga algicola | Q265 | T436 | N438 | H442 |
| Myroides odoratimus | Q265 | T436 | N438 | H442 |
| Novosphingobium sp. PP1Y | Q263 | T434 | N436 | H440 |
| Thalassolituus oleivorans | Q263 | T434 | N436 | H440 |
| Frateuria aurantia | Q259 | T430 | N432 | H446 |
| Ochrobactrum rhizosphaerae | Q260 | T431 | N433 | H447 |

For construction of OhyA mutants, the following primers were used according to the manufacturer's manual (Stratagene QuikChange™ site-directed mutagenesis protocol). 25 μL of two separate PCR reactions containing forward and reverse primers, respectively, were prepared (Table 2). After 5 cycling steps, PCR reactions were combined, and PCR was continued for 20 additional cycles according to the manual. Mutated plasmids were verified by DNA sequencing of the coding regions of the constructs.

TABLE 2
primers used for construction of OhyA mutants.
The bold letters indicate the mutated codon.

| Primer/ | | SEQ |
|---|---|---|
| sequence name | sequence 5′ to 3′ | ID NO: |
| Fw(OhyA_Q265A) | GTTTCCGAAGTACAATGC | 3 |
| | ATATGACACGTTTGTC | |
| Rv(OhyA_Q265A) | GACAAACGTGTCATATGC | 4 |
| | ATTGTACTTCGGAAAC | |
| Fw(OhyA_T436A) | TGGTTGATGAGCTTTGCG | 5 |
| | TGCAATCGCCAGCCG | |
| Rv(OhyA_T436A) | CGGCTGGCGATTGCACGC | 6 |
| | AAAGCTCATCAACCA | |
| Fw(OhyA_N438A) | GATGAGCTTTACCTGCGC | 7 |
| | ACGCCAGCCGCATTTCC | |
| Rv(OhyA_N438A) | GGAAATGCGGCTGGCGTG | 8 |
| | CGCAGGTAAAGCTCATC | |
| Fw(OhyA_H442A) | CTGCAATCGCCAGCCGGC | 9 |
| | CTTCCCGGAGCAGCCGG | |
| Rv(OhyA_H442A) | CCGGCTGCTCCGGGAAGG | 10 |
| | CCGGCTGGCGATTGCAG | |
| Fw(OhyA_T436A/ | GATGAGCTTTGCGTGCGC | 11 |
| N438A) | ACGCCAGCCGCATTTCC | |
| Rv(OhyA_T436A/ | GGAAATGCGGCTGGCGTG | 12 |
| N438A) | CGCACGCAAAGCTCATC | |

For purification of the recombinant OhyA, cell pellets were resuspended in 50 mM HEPES, pH 7.4, containing 10 mM imidazole. Cells were lysed by ultrasonication for 4 min with a Sonifier® 250 (Branson, Danbury, Conn.) setting the duty cycle to 80% and the output control to level 8. Cell free extract (CFE) was separated from the total cell lysate (TCL) by centrifugation for 35 min at 48,300×g and 4° C., and was filtered through 0.22 μm filters (Millipore, Bedford, Mass.) prior to loading it onto a pre-equilibrated self-packed Ni-NTA affinity chromatography column (GE Healthcare, United Kingdom). Prior to any further analyses, purified OhyA was incubated with a 10-fold molar excess of FAD over night at 4° C. Unbound FAD was removed from the protein aliquot by buffer exchange via PD-10 desalting columns (GE Healthcare, United Kingdom) according to the recommended protocol.

UV-Visible (UV-Vis) absorption spectra of wt-OhyA and mutant OhyA Q265A/T436A/N438A were recorded at a spectral range from 250 nm to 1,000 nm on a Specord 205 double-beam spectrophotometer (Analytik Jena AG, Germany) using quartz cuvettes with a path length of 1 cm. Spectral measurements were performed in 50 mM HEPES, pH 7.4, containing 50 mM NaCl. The binding of FAD to purified OhyA was determined by calculating the concentration of the protein based on the ϵ₂₈₀ of 111,115 M⁻¹ cm⁻¹ for FAD-loaded enzyme, and the previously measured ϵ₄₈₀ of 8,074 M⁻¹ cm⁻¹ of FAD non-covalently linked to the OhyA structure.

Free fatty acids were identified and analyzed by gas chromatography-mass spectrometry (GC-MS) and comparison of derived mass fragmentation spectra to authentic standards. A HP-5 column (crosslinked 5% Ph-Me Siloxane; 30 m length, 0.25 mm in diameter and 0.25 μm film thickness) on a Hewlett-Packard 6890 Series II GC equipped with a mass selective detector was used. Sample aliquots of 1 μL were injected in split mode (split ratio 30:1) at 240° C. injector temperature and 290° C. detector temperature with N₂ as carrier at a flow rate set to 36 cm s⁻¹ in constant flow mode. The temperature program was as follows: 100° C. for 1 min, 15° C. min⁻¹ to 300° C., hold for 5 min. The total run time was 19.33 min. The mass selective detector was operated in a mass range of 50-400 amu at an electron multiplier voltage of 1765 V. Results were evaluated with the GC-MS Data Analysis software (Agilent Technologies, Austria).

### Example 2: Whole Cell Biotransformation of OhyA Mutants Expressed in E. coli

OhyA was recombinantly expressed in E. coli. First, a pre-culture was inoculated with E. coli BL21 Star (DE3) cells harboring pMS470-HISTEV-OhyA wild type enzyme or variants, and was grown in LB supplemented with 100 μg mL⁻¹ ampicillin at 28° C. and 130 rpm overnight. Main cultures were inoculated to an OD₆₀₀ of 0.1 in auto induction medium (AIM)—Terrific Broth Base including Trace elements (Formedium, UK) containing 100 μg mL⁻¹ ampicillin. Recombinant protein was expressed at 28° C. and 130 rpm for 22 h. Cells were harvested by centrifugation for 10 min at 4,400×g and 22° C. and were instantly used for whole cell biotransformation or were frozen at −20° C. until protein purification.

For bioconversion assays of oleic acid (OA) and OA derivatives, i.e. substrates (1) to (10), 50 OD₆₀₀ units of thus prepared cells, which are corresponding to a cell dry weight of 50 mg, were resuspended in 50 mM HEPES, pH 6.0, supplemented with 100 mM glucose and 0.2 mM FAD in Pyrex® glass culture tubes (Corning, N.Y.). Biotransformation at 1 mL scale were started by adding substrate to a final concentration of 2 mM from an ethanolic stock solution (100 mM). n-pentadecanoic acid (1 mM) was used as internal standard. The reactions were conducted in the presence of 2% (v/v) of ethanol as co-solvent at 30° C. and shaking at 150 rpm at a defined angle of the Pyrex® tubes (55°). Biotransformation were performed for 22 h or 96 h. The assays were quenched by acidification to pH 2.0 with 0.12 M HCl, and fatty acid derivatives were extracted twice with 2 mL of ethyl acetate while agitating on a Vibrax VXR basic shaker (IKA, Germany) for 30 min. The suspension was centrifuged for 5 min at 2,900×g and 22° C. to improve the separation of the phases. Combined organic phases were concentrated under a N₂ stream. Fatty acid derivatives were silylated with 10 μL of pyridine and 50 μL of N,O-bis(trimethylsilyl)trifluoroacetamide (BSTFA). After incubation for 30 min at 500 rpm, extracts were diluted with 200 μL of ethyl acetate and analyzed by GC-MS.

### Example 4: Reduction of the FAD Cofactor and Anaerobic In Vitro Conversions

For reduction of the enzyme-bound flavin cofactor, wt-OhyA and mutant Q265A/T436A/N438A were diluted in 50 mM HEPES, pH 7.4, containing 50 mM NaCl, 5 mM EDTA, 1 μM 5-deazariboflavin and 4 μM methyl viologen to give an absorption of approximately 0.2 AU at 440 nm. The solution was transferred to a cuvette and oxygen was removed by incubation for at least 2 h in a glove box. Afterwards, the cofactor was reduced by anaerobic photoreduction or chemical reduction with dithiothreitol (DTT). For anaerobic photoreduction, the FAD cofactor was reduced for 150 min by irradiation with a conventional LED (Luminea NC-691; Pearl GmbH, Buggingen, Germany; 10 W, 5400 K). During photoreduction, the cuvette was cooled to 10° C. to compensate for the heat generated by the light source. For chemical reduction, a 100-fold molar excess of DTT was added to the samples. Activity assays with reduced and oxidized OhyA were performed under anaerobic conditions. Assay reactions contained 0.15 mg mL⁻¹ of reduced or oxidized OhyA and 2 mM of substrates (1), (6) and (7) at a 1 mL scale in 50 mM HEPES, pH 6.0, in presence of 2% (v/v) of ethanol. Conversions of OA, i.e. substrate (1), were incubated for 10 min at 21° C. under manual shaking in a glove box using n-pentadecanoic acid as internal standard. Reactions with OA esters, substrates (6) and (7) were incubated over night at 25° C. and 150 rpm at a 55° angle in Pyrex® glass culture tubes. The reactions were then stopped for GC-MS analyses conducted as described herein.

### Example 4: Hydration of OA Derivatives in a Semi-Preparative Scale with E. coli Whole Cells

OA derivatives, i.e. substrates (3) to (10) were hydrated in a semi-preparative scale. 20-150 mg of non-physiological substrates were converted in 1 mL scale whole cell bioconversions. Each reaction contained 200 mg of E. coli cells in Pyrex® glass culture tubes after over-expression of OhyA Q265A/T436A/N438A, resuspended in 50 mM HEPES, pH 6.0, containing 100 mM glucose and 0.2 mM FAD. Biotransformation were incubated for 96 h at 30° C. and 150 rpm at a defined angle of the Pyrex® tubes (55°). After quenching by acidification to pH 2.0 with 0.12 M HCl, the suspensions were extracted with ethyl acetate (3×2 mL for 30 min) with intermittent centrifugation for 5 min at 2,900×g and 22° C. to improve the phase separation. The organic phases were quantitatively collected and concentrated under a stream of N₂. The results are shown in Table 3.

TABLE 3
Isolated yield of OA derivatives and hydrated
products from E. coli cell extracts.

| | Starting | Starting material, | Hydrated product, |
|---|---|---|---|
| Entry | material/mg | recovered/mg | isolated/g |
| 3 | 20 | | |
| 4 | 60 | | |
| 5 | 20 | 7.8 | 6.3 |
| 6 | 80 | 62.4 | 5.4 |
| 7 | 80 | 60.9 | 10.4 |
| 8 | 80 | 59.1 | 1.6 |
| 9 | 60 | | |
| 10 | 150 | | |

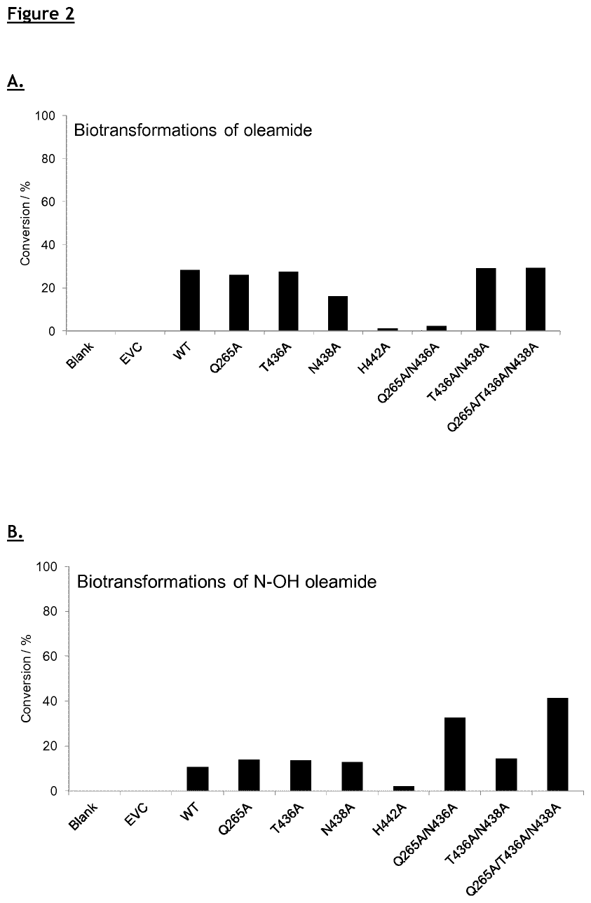

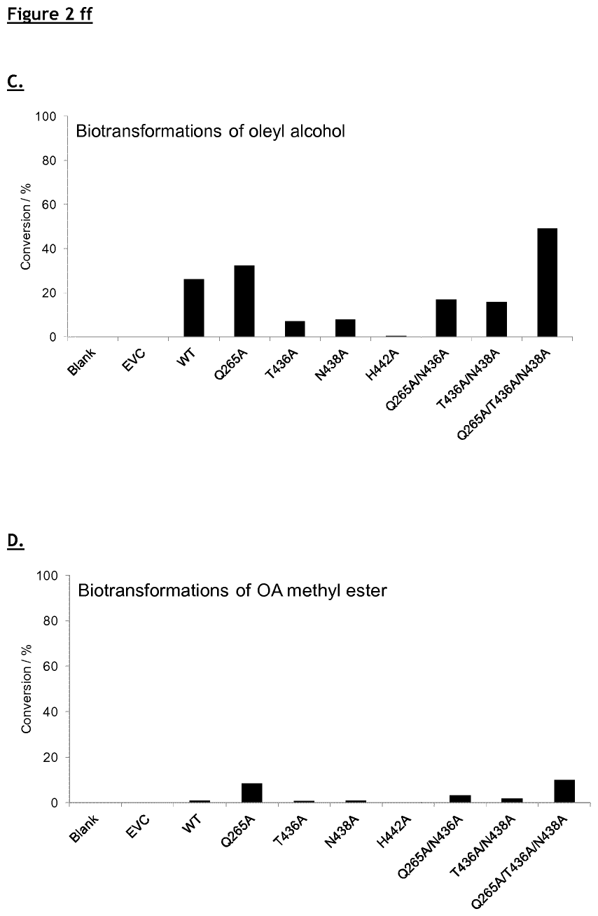

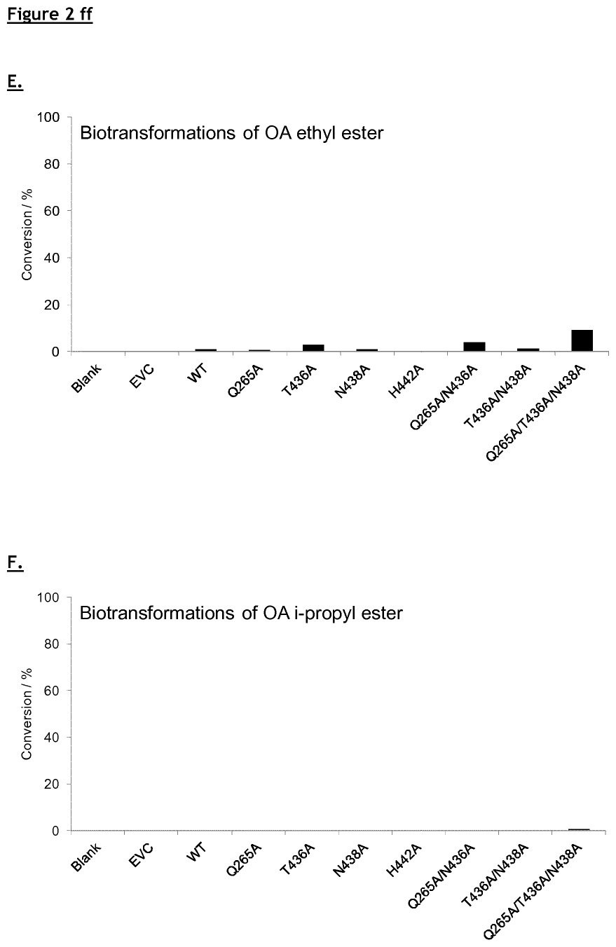

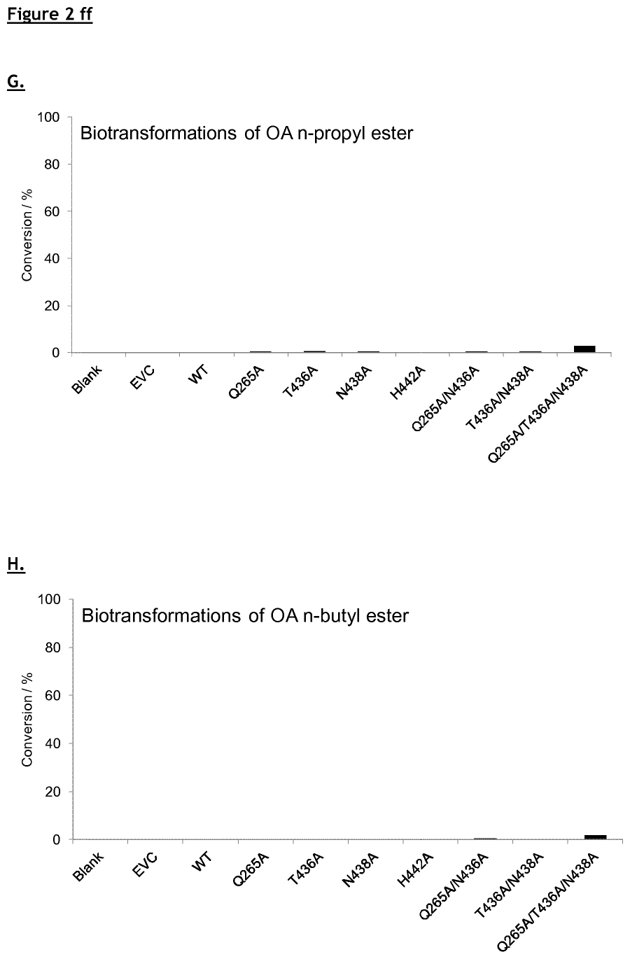

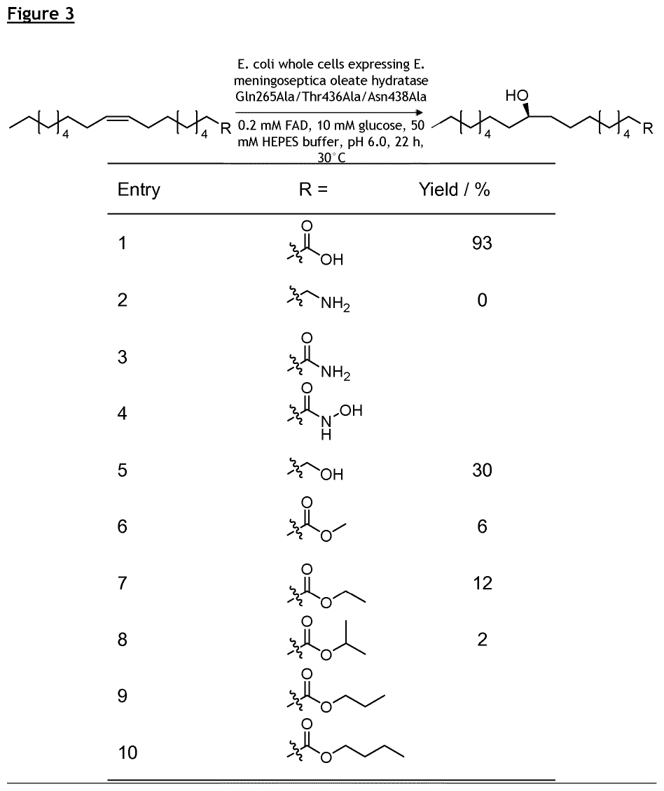

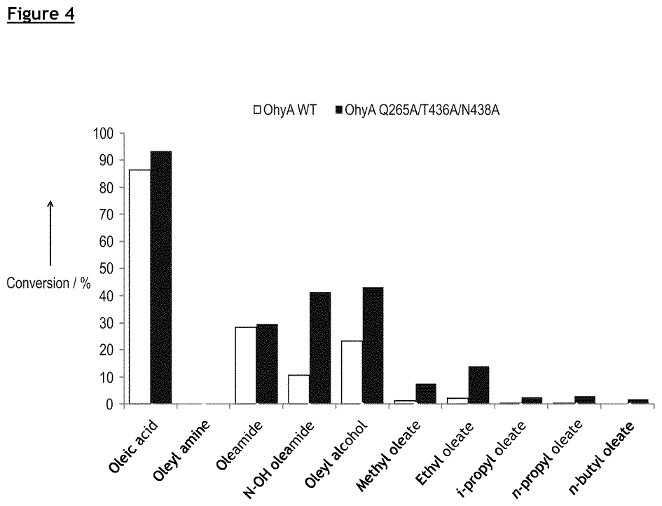

Bioconversions of substrates (1) to (10) were performed with whole E. coli cells—an E. coli empty vector control (EVC) and a biotransformation with cells after over-expression of OhyA are overlaid—with substrate (1) being oleic acid, substrate (3) being oleamide, substrate (4) being N-hydroxy oleamide to be converted into N,10-dihydroxyoctadecanamide, substrate (5) being oleyl alcohol to be converted into 1,10-octadecanediol, substrate (6) being OA methyl ester (methyl oleate) to be converted into 10-hydroxy octadecanoic acid methyl ester, substrate (7) being OA ethyl ester (ethyl oleate) to be converted into 10-hydroxy octadecanoic acid ethyl ester, substrate (8) being OA isopropyl ester (i-propyl oleate) to be converted into 10-hydroxy octadecanoic acid isopropyl ester, substrate (9) being OA n-propyl ester (n-propyl oleate) to be converted into 10-hydroxy octadecanoic acid n-propyl ester, and substrate (10) being OA n-butyl ester (n-butyl oleate) to be converted into 10-hydroxy octadecanoic acid n-butyl ester (not shown). The OA-derived hydroxamic acid, i.e. substrate (4), and the hydrated reaction product were both detected as the respective isocyanates after a Lossen rearrangement occurring under GC-MS analysis conditions. Moreover, conversion of substrate (4) with the E. coli EVC and the strain expressing OhyA led to the unexpected formation of oleamide, i.e. substrate (3), with a subsequent hydration to 10-hydroxy octadecanamide only in OhyA biotransformation. Since the substrate (4) was initially oleamide-free, one must assume that the oleamide was formed by degradation of the substrate (4) in E. coli. Conversion rates using substrates (3) to (10) are shown in FIG. 2. Numbers normalized for biomass of whole cell E. coli biocatalysts are shown in Table 4.

TABLE 4
Apparent hydration activity normalized for biomass of whole cell
E. coli biocatalysts harboring OhyA wild type (WT) and mutant
OhyA Q265A/T436A/N438A enzymes for the regio- and stereoselective
hydration of oleic acid and derivatives thereof.

| | | mU g⁻¹ CDW | | |
|---|---|---|---|---|
| | OhyA | OhyA | Abs. conf. | e.e. |
| Entry | WT | Q265A/T436A/N438A | at C-10 | [%] |
| 1 | 26.3 ± 0.2 | 28.4 ± 0.3 | R | >99 |
| 2 | — | — | R | >99 |
| 3 | 11.1 ± 0.7 | 10.7 ± 0.3 | R | >99 |
| 4 | 3.4 ± 0.7 | 13.4 ± 2.5 | R | >99 |
| 5 | 8.4 ± 2.1 | 15.5 ± 1.0 | R | >99 |
| 6 | 0.3 ± 0.1 | 2.5 ± 0.2 | R | >99 |
| 7 | 0.2 ± 0.1 | 1.7 ± 0.1 | R | >99 |
| 8 | 0.1 ± 0.1 | 1.5 ± 0.1 | R | >99 |
| 9 | 0.2 ± 0.1 | 0.9 ± 0.1 | R | >99 |
| 10 | 0.1 ± 0.1 | 0.8 ± 0.1 | R | >99 |

### Example 5: Hydration of OA Derivatives in a Semi-Preparative Scale with Cell Free Extracts (CFE)

In vitro activity assays were performed with 2 mg of E. coli CFE after recombinant protein expression in Pyrex® glass culture tubes. CFE was incubated with 2 mM substrates (1) to (10) in 1 mL of 50 mM HEPES, pH 6.0, and 2% (v/v) of ethanol. Assays were shaken over night at 25° C. and 150 rpm in the presence of 1 mM n-pentadecanoic acid as internal standard. Conversions were quenched and fatty acids were extracted and derivatized as described in Example 2.

Anaerobic in vitro hydration reactions of substrate (1) and OA esters, substrates (6) and (7), were performed with purified wt-OhyA and mutant OhyA Q265A/T436A/N438A after reduction of the FAD cofactor. Reactions containing substrate (1) were quenched after 10 min, and reactions containing substrate (6) or (7) were quenched after overnight incubation (not shown). In conversions of substrate (6), we observed a peak at the expected retention time of the hydrated product after incubation with authentic OA standard, wt-OhyA and mutant OhyA Q265A/T436A/N438A only in the case of the variant as proven by GC-MS analysis (not shown).

1. A modified enzyme having fatty acid hydration activity, particularly activity towards hydration of oleic acid derivatives, comprising one of more amino acid substitution(s) at (a) position(s) corresponding to residues selected from 265 and/or 436 and/or 438 and/or 442 in the polypeptide according to SEQ ID NO: 1.

2. A modified enzyme according to claim 1, wherein the oleic acid derivate is a non-natural oleic acid derivative, preferably an oleic acid ester.

3. A modified enzyme according to claim 1, wherein the amino acid substitution is selected from the group consisting of Q265A, T436A, N438A, H442A, and combinations thereof.

4. A modified enzyme according to claim 3, wherein the amino acid substitution is selected from a combination of Q265A/T436A/N438A.

5. A modified enzyme according to claim 1 capable of hydrating an oleic acid derivative with an enantiomeric excess (ee) of at least 98%.

6. A modified enzyme according to claim 1 wherein the hydration activity is at least 2-fold higher compared to a wild-type enzyme.

7. A modified enzyme according to claim 1 capable of catalyzing the hydration of oleic acid derivatives independent of co-factors, preferably independent of FAD or DTT.

8. A process for hydration of oleic acid derivatives using a modified enzyme according to claim 1.

9. A process according to claim 8, wherein the process is conducted in whole cell biotransformation.

10. A process according to claim 8, wherein the modified enzyme is expressed in E. coli.


SEQUENCE LISTING (12 sequences)

SEQ ID NO: 1 - protein, 646 aa, Elizabethkingia meningoseptica
MNPITSKFDKVLNASSEYGHVNHEPDSSKEQQRNTPQKSMPFSDQIGNYQRNKGIPVQSYDNSKIYIIGSGIAGMSAAYYFIRDGHVPAKNITFLEQLHIDGGSLDGAGNPTDGYIIRGGREMDMTYENLWDMFQDIPALEMPAPYSVLDEYRLINDNDSNYSKARLINNKGEIKDFSKFGLNKMDQLAIIRLLLKNKEELDDLTIEDYFSESFLKSNFWTFWRTMFAFENWHSLLELKLYMHRFLHAIDGLNDLSSLVFPKYNQYDTFVTPLRKFLQEKGVNIHLNTLVKDLDIHINTEGKVVEGIITEQDGKEVKIPVGKNDYVIVTTGSMTEDTFYGNNKTAPIIGIDNSTSGQSAGWKLWKNLAAKSEIFGKPEKFCSNIEKSAWESATLTCKPSALIDKLKEYSVNDPYSGKTVTGGIITITDSNWLMSFTCNRQPHFPEQPDDVLVLWVYALFMDKEGNYIKKTMLECTGDEILAELCYHLGIEDQLENVQKNTIVRTAFMPYITSMFMPRAKGDRPRVVPEGCKNLGLVGQFVETNNDVVFTMESSVRTARIAVYKLLNLNKQVPDINPLQYDIRHLLKAAKTLNDDKPFVGEGLLRKVLKGTYFEHVLPAGAAEEEEHESFIAEHVNKFREWVKGIRG

SEQ ID NO: 2 - DNA, 1974 bp, Elizabethkingia meningoseptica
atgcatcaccatcaccatcaccatcaccatcacaacccaatcaccagcaaattcgacaaagtcctgaacgcatccagcgagtacggccacgttaatcacgaaccggatagcagcaaagagcagcaacgcaacaccccgcagaagtccatgccatttagcgatcaaatcggcaactatcaacgtaacaaaggtattccggttcagagctatgataattcgaagatttacatcattggttctggtattgcgggtatgtcggctgcgtactacttcatccgtgacggtcacgttccggcgaagaacatcacgttcctggagcaactgcacattgatggcggctctctggatggtgctggcaacccgaccgacggctatatcatccgtggtggtcgtgaaatggatatgacctacgagaacctgtgggatatgttccaggatattccggcgctggagatgccggcaccgtatagcgttctggatgaatatcgtctgattaatgacaacgatagcaattacagcaaagcacgtctgatcaacaataagggcgaaatcaaggacttcagcaagtttggtctgaataagatggaccagctggccatcatccgtctgttgctgaaaaacaaagaagagctggacgacttgaccattgaggactacttctctgagagctttctgaaaagcaatttctggacgttttggcgcacgatgttcgcgtttgagaactggcatagcctgttggaactgaagctgtacatgcaccgcttcctgcacgccattgacggtctgaacgacctgagcagcctggtgtttccgaagtacaatcaatatgacacgtttgtcacgccgctgcgtaaattcctgcaagaaaagggtgttaacatccacttgaataccttggtcaaggatctggatattcacatcaataccgagggtaaagtcgtcgagggcatcattaccgagcaagacggtaaagaggtaaagattccggtgggtaagaatgactatgttatcgtgacgaccggttccatgaccgaggacacgttttacggtaacaacaaaaccgcaccgatcattggtatcgacaatagcactagcggtcagagcgctggctggaaactgtggaagaacctggctgccaagagcgaaatcttcggcaagccggagaaattctgtagcaatattgagaaatccgcgtgggaaagcgcgaccctgacgtgtaaaccttccgcgttgatcgacaaactgaaagaatattcggtcaacgacccgtacagcggtaagaccgtgaccggcggtatcattactatcaccgatagcaactggttgatgagctttacctgcaatcgccagccgcatttcccggagcagccggatgacgtcctggtgctgtgggtgtatgcgctgtttatggataaagaaggtaactacattaagaaaaccatgctggagtgcaccggtgatgagattttggcggagctgtgttaccatctgggcattgaagatcagctggaaaatgtgcagaagaatacgattgttcgcaccgcattcatgccgtatatcacgagcatgtttatgccacgtgccaaaggtgaccgccctcgtgtggtcccggaaggttgcaaaaacctgggcctggttggtcaatttgtggaaacgaacaatgacgtcgtgtttacgatggaatctagcgttcgcacggcccgtattgcggtgtacaagttgctgaatctgaacaagcaggtgccggacattaatccgctgcaatacgatattcgccatctgctgaaagcggcaaagaccctgaatgatgacaaaccgttcgtgggcgaaggcttgctgcgtaaggttctgaaaggcacctattttgagcacgttctgcctgcgggtgcagcggaagaagaagagcatgagagcttcattgcggaacatgttaacaagttccgtgagtgggtcaagggtatccgtggctaataa

SEQ ID NO: 3 - DNA, 34 bp, Artificial Sequence
  primer
gtttccgaagtacaatgcatatgacacgtttgtc

SEQ ID NO: 4 - DNA, 34 bp, Artificial Sequence
  primer
gacaaacgtgtcatatgcattgtacttcggaaac

SEQ ID NO: 5 - DNA, 33 bp, Artificial Sequence
  primer
tggttgatgagctttgcgtgcaatcgccagccg

SEQ ID NO: 6 - DNA, 33 bp, Artificial Sequence
  primer
cggctggcgattgcacgcaaagctcatcaacca

SEQ ID NO: 7 - DNA, 35 bp, Artificial Sequence
  primer
gatgagctttacctgcgcacgccagccgcatttcc

SEQ ID NO: 8 - DNA, 35 bp, Artificial Sequence
  primer
ggaaatgcggctggcgtgcgcaggtaaagctcatc

SEQ ID NO: 9 - DNA, 35 bp, Artificial Sequence
  primer
ctgcaatcgccagccggccttcccggagcagccgg

SEQ ID NO: 10 - DNA, 35 bp, Artificial Sequence
  primer
ccggctgctccgggaaggccggctggcgattgcag

SEQ ID NO: 11 - DNA, 35 bp, Artificial Sequence
  primer
gatgagctttgcgtgcgcacgccagccgcatttcc

SEQ ID NO: 12 - DNA, 35 bp, Artificial Sequence
  primer
ggaaatgcggctggcgtgcgcacgcaaagctcatc